(19) United States
(12) Patent Application Publication
(10) Pub. No.: US 2006/0181677 A1
(43) Pub. Date: Aug. 17, 2006

(54) Method or apparatus for inhibiting myopia development in humans

(76) Inventor: Katrina Schmid, Queensland (AU)

Correspondence Address: CANTOR COLBURN, LLP, 55 GRIFFIN ROAD SOUTH, BLOOMFIELD, CT, 06002, US

(21) Appl. No.: 10/535,085

(22) PCT Filed: Nov. 14, 2003
(86) PCT No.: PCT/AU03/01527

§ 371 Date: May 13, 2005

(30) Foreign Application Priority Data
Nov. 14, 2002 (AU) 2002952682

(51) Int. Cl. A61B 3/14

(52) U.S. Cl. 351/206

(57) ABSTRACT

A method of inhibiting myopia by exposing a person to a strobing or flickering light or pattern at a prescribed frequency and having a prescribed wavelength for a prescribed period.

### FIELD OF THE INVENTION

This invention relates to a method and apparatus for inhibiting the development of myopia in humans.

### BACKGROUND OF THE INVENTION

Myopia is a refractive eye disorder that affects a large segment of the population (30% in Australia, up to 90% in Asia). In particular it is characterised by a normal ability to see nearby objects but a reduced ability to see objects at a far distance. Thus the colloquial term for this condition is nearsightedness or shortsightedness.

This condition can have an onset either during childhood, especially from the ages of 6 to 14 years, or young adulthood (15 to 25 years) and typically worsens, particularly as a person grows through adulthood. The person's vision becomes increasingly blurry when focusing on distant objects, requiring increasingly stronger optical correction over time.

There are various anatomical explanations for the presence of myopia. These include the eyeball developing with a greater than normal length, possibly due to developing an enlarged vitreous chamber, alternately the cornea or the lens may be too strongly powered. The most common cause is a longer than normal eye.

These developments result in the eye not needing to accommodate to focus on near objects and create a blurry image on the retina when focusing on more distant objects. Further it is suggested that both genetic and environmental factors are important in myopia development with prolonged near work being associated with myopia.

Animal models have shown that abnormal visual experience can lead to myopia. For example, translucent diffusers placed over the eyes of animals causes them to develop myopia.

In “Experimental Myopia in Cats Reared in Strobic illumination” (Cremieux, Orban, Duysen, Amblard and Kennedy), experiments on cats have shown that myopia can be induced by subjecting kittens to low frequency strobing lights (˜2 Hz) for more than 4 hours per day. This suggests that test subject animals can be prepared for myopia studies by exposing young animals to low frequency strobic illumination while they are developing their vision.

A popular way to compensate for myopia is to use concave lenses, for example in eyeglasses or contact lenses. The concave lens shifts the image plane to be coincident with the retina and thus brings the distant objects into clearer focus. A problem with these lenses is that they do not stop the myopia from developing and as the eye continues to elongate, stronger and stronger lenses are required and vision gradually worsens.

Another form of correcting myopia is to operate on the cornea using refractive laser surgery techniques. This remedy is expensive and the long term effects are not yet known. Furthermore this treatment is only available to adults with stabilised myopic eyes and further operations may be required if the refractive error changes in the future. Surgical correction of the myopia can also result in a slight reduction in best vision and does not treat the cause of the myopia (ie. an elongated eye).

Another costly remedy is for the myopic individual to take drugs and eye drops (for example pirenzipine) to combat the myopia. There are not currently any drops that are known to effectively inhibit myopia development. Once again, the long term effects of these remedies are unknown and it is a costly solution to the problem involving continual prescriptions and health risks. Further, the eye drops may include side effects of dilating the pupil and reducing the focusing ability of the patient.

There is a need for a low cost, non-invasive treatment that assists in the retardation or inhibition of the development of myopia, especially one that is safe for use on children during the onset of myopia.

### OBJECT OF THE INVENTION

It is an object of the invention to overcome or alleviate one or more of the above problems or to provide the consumer with a useful commercial choice.

### DISCLOSURE OF THE INVENTION

In one form, although it need not be the only or indeed the broadest form, the invention resides in a method of inhibiting myopia development in a human subject including the steps of:

prescribing a frequency and exposure time of a strobing or flickering light or pattern to reduce the rate of myopia development for the subject;

treating the subject with a strobing or flickering light or pattern at the prescribed frequency and exposure time.

Preferably the treatment is repeated as required, such as daily.

Preferably, the method also includes the step of measuring the myopia of the subject.

By ‘inhibiting’ it is meant that the treatment reduces the advance of existing myopia and may prevent development of myopia if treated before onset.

In another form, the invention resides in a method of inhibiting myopia development in human subjects including the step of:

exposing the eyes of a subject to light flashing at a frequency in the range of 1 to 60 Hz for at least ten minutes per treatment.

Preferably the treatment occurs each day or each alternate day.

Preferably, the method includes a feedback loop for adjusting the treatment in response to the effectiveness of the treatment in terms of measured progress of the subject.

Preferably, the treatment is applied during daylight hours.

The treatment will preferably involve visible light (excluding ultraviolet and infrared) and may exclude short wavelengths (blue light).

In another form, the invention resides in an apparatus for inhibiting myopia developments in human subjects comprising:

a strobable light;

a means of adjusting a frequency at which the light strobes;

a means of adjusting a period of time over which the light strobes; and

wherein said light strobes at a desired frequency for a desired time period.

The apparatus may further comprise a feedback means of measuring myopia and making an adjustment to the period of time and the frequency the light strobes in response to the measured myopia.

Suitably, the apparatus operates at a frequency in the range 1 to 60 Hz.

Most preferably, the frequency used is in the range 5 to 20 Hz.

Generally the frequency used will compensate for the frequency of the background lighting.

Suitably, the time period will last for at least five minutes each day, or preferably ten or twenty minutes each day.

Most preferably the treatment will be applied for 5 or 10 minute periods every hour over a 2 to 10 hour period. 20 Generally the intensity of the light used will compensate for the intensity of the background lighting.

Most preferably, the wavelength of the light will be about 550 nm.

Suitably the wavelength of light will be selected to compensate for the wavelength of the background light.

Preferably, the apparatus may further include a base.

Preferably the base will be in the form of eyeglass frames with the light located near the hinge.

In another form the base will be mountable to a table.

The base may be in the form of a lamp stand.

### BRIEF DESCRIPTION OF THE DRAWINGS

Embodiments of the invention will now be described with reference to the accompanying drawings in which:

FIG. 1 shows a flow diagram of the steps involved in the invention;

FIG. 2 shows a diagram of the invention mounted to a eyeglass base;

FIG. 3 shows a diagram of the invention mounted to a lamp stand base:

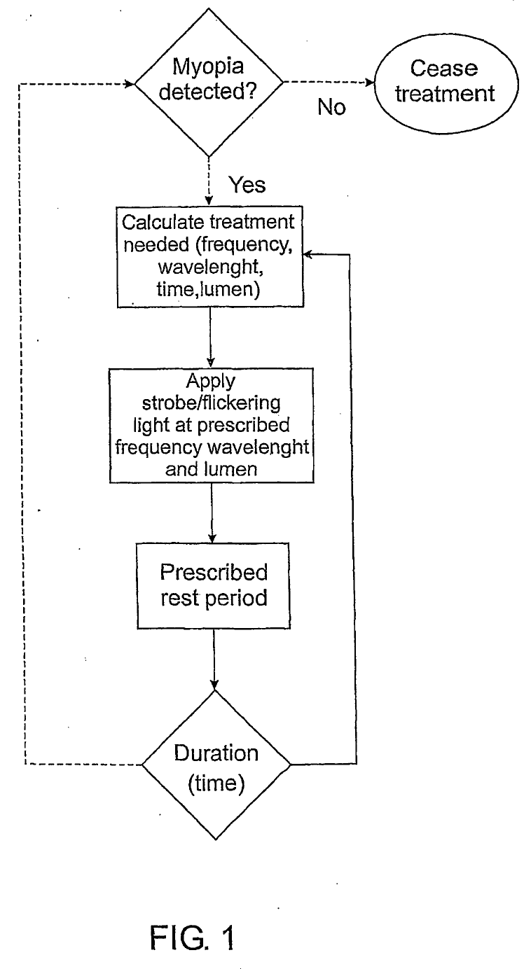

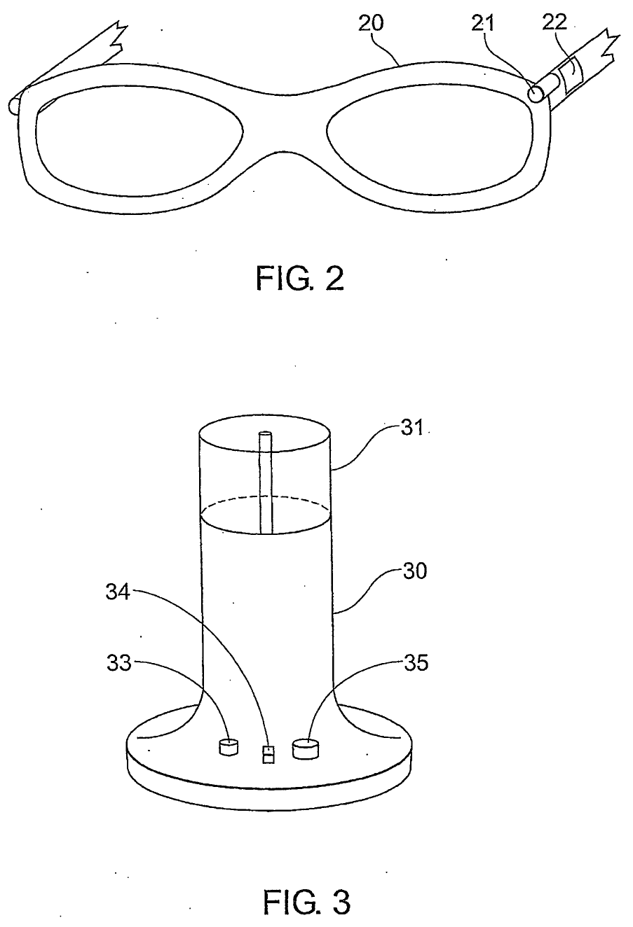

### DETAILED DESCRIPTION OF THE DRAWINGS

The first step in treating myopia is to assess the subject for their current condition. There are various means for testing myopia including using an opthalmoscope, refractometer, infrared retinoscopy, A scan ultrasound, or flicker ERG, to test the reflection from the retina, with myopia being diagnosed when the subject's refraction is measured to be negative (typically in the range of 0 D to −10 D), with more negative values representing more severe myopia. Once the subject is identified as myopic, the extent of myopia ran be assessed to determine the best treatment.

Once the extent of myopia is known, the next step is to determine a specific treatment for the subject. In particular, specific frequency ranges and durations for treatment will target the particular myopia. The optimal frequency may vary and faster progression rates may require higher frequencies and longer duration of treatment.

Flicker ERG may be utilised to determine the optimal flicker frequency and/or stimulus pattern. Subjective responses of flicker perception and/or the critical fusion frequency may also be utilised.

Factors such as background lighting frequency, background lighting wavelength and background lighting intensity may be compensated by the strobe light for the best results. For example, if background lighting is incandescent it has little 50 Hz flicker and provides light with a wavelength in the yellow range around 600 nm, while fluorescent lighting may have 100 Hz flicker and much higher colour temperatures, in the blue region of the spectrum.

White light stimulates the maximum number of cone photoreceptors in the eye, as it activates the long, mid and short wavelengths. Hence the method or apparatus will be most effective for stimulating improved vision when the subject is exposed to white light. Thus when the background lighting is blue, as in halogen lighting, the applied lighting must compensate for the lack of red and green wavelengths. Alternatively, when the background lighting is yellow, as in incandescent lighting, the compensating light must be more in the blue/green bandwidth range.

Intensity of the background lighting will also need to be considered when prescribing a strobe light. The optimal intensity for treatment is 1000 to 1300 lumen. Thus if the background lighting is dimmer than this, the treatment may not be as effective as the strobing light will be distracting and visually disturbing. When the background lighting is at the high end of this intensity range, the strobe light can be brightened to complement it.

Additionally the frequency of the strobe light will need to be calculated to compensate for the background lighting frequency, for example, the 50 Hz of halogen lighting or the lack of flicker in incandescent lighting. A typical frequency for the device would be within the range 5 to 20 HZ, but it would be possible to use frequencies between 1 to 60 Hz to the same effect. The impact of strobe lighting on subjects would need to be considered before prescribing the treatment, as it is understood epilepsy can be triggered by certain frequencies and thus the treatment may be of lesser use for a subject with epileptic tendencies.

After characterising the myopia and calculating the treatment parameters, the next step is to prescribe a device for the subject to use for treatment. For example a light emitting diode (LED) could be positioned on a base worn on the subject's head during reading. The diode would emit light at a particular wavelength, with a programmed frequency, programmed illumination/dimness and programmed duration determined specifically for the subject.

The light emitting diode device would include means for adjusting the frequency, illumination and duration of treatment as required for the subject. The diodes are replaceable to provide for different wavelengths of light for treatment as required.

Current microprocessor technology allows the production of small, application specific integrated circuits which would be suitable for providing the required control of wavelength, intensity, pulse frequency and pulse duration.

Another embodiment would be to use a strobe light on a base to emit the light for treatment. Once again, this lamp would need to include a means for adjusting the frequency, illumination and duration of the light to be used for treating subjects.

The base as illustrated in FIG, 2 and FIG. 3 could comprises a portable structure such as spectacle frames (20) or other head attachments to allow the light, such as a LED (21), to be positioned close to the eye and controlled by a microprocessor (22). Additionally the base could be a more solid structure, such as a lamp base (30), formed to rest on a desk or table during use. In this form the light source would be a strobe lamp (31) supported on the base (30) and having a control to adjust the frequency (33), on/off toggle switch (34) and an adjustable timer (35).

The optimal delivery of the strobe treatment would be for 10 minutes per hour throughout the day. In practical application, it may also be provided in a single duration once per day. For children at school, an effective treatment would be during an hour of reading after school.

Although the preferred treatment delivery modality is a strobing light as described above, other temporally modulated flickering targets may be effective. For instance, the strobing light may be replaced by a flickering pattern on a screen, such as a computer monitor or small hand-held display. In this embodiment the pattern is made up of a grid of lines, squares, or other shapes. The pattern has areas of low luminance (black) and high luminance (white) which alternate at a predetermined frequency. This delivery method may be more suitable for older children who spend a significant amount of time looking at a computer screen. The effect is essentially the same as the strobing light but is delivered directly from the viewing are. The Treatment may be delivered from a small section of the screen while other programs are running or may be part of a separate treatment program that runs at predetermined times.

Similarly, the treatment may be delivered from a television screen while watching television programs. In this embodiment a small set-top box delivers a television frequency signal in-line with the received television signal. The set-top box is programmed to provide a ‘test pattern’ type signal in one corner of the screen. As with the strobing light embodiment the ‘test pattern’ flickers at a pre-determined frequency for a pre-determined period of time.

The final step in the iterative process is a feedback loop, where myopia is remeasured and treatment is recalculated. The success of the treatment will be measurable as a reduction in the myopia of the subject. As the myopia reduces the treatment required will need to be adjusted with frequencies reduced and duration decreased. This would be achieved by adjusting the frequency and the duration.

A professional with the measurement methods described for diagnosing the myopia can perform the measurement of the myopia, at a designated time after treatments. Additionally a feedback mechanism can be included with the device, which automatically adjusts the treatment. Once the reduced myopia is measured, a new program of treatment will be calculated considering new frequency and new duration required.

A feedback mechanism for automatically adjusting the treatment would measure the electrical signals from the retinal output and calculate the new required parameters, or a subjective psychophysical equivalent could be used.

Treatment of subjects is measured as a reduction in the rate of growth of myopia with an expected reduction of 50%. This treatment is an iterative process with the measurements providing a feedback mechanism so the treatment can be controlled as required. At a predetermined point of measured myopia progression, such as −0.25 D, treatment may no longer be needed and the subject should be monitored for future regression in vision.

Recent scientific experiments on animals have suggested that exposing the eyes of test subjects to flashing lights at a certain frequencies can cause myopia to develop. This has been useful in providing animal subjects with myopia so that various remedies can be tested on them. This research is in conflict with the invention herein described, as flashing lights are being used to treat existing myopia rather than cause it.

As domestic lighting is commercially available in specific packaged forms, specific compensating lights can be prepared for use with lighting available in the market. For example if the background light is an incandescent 100 W globe, the compensating frequency, wavelength and luminosity can be predetermined.

It should be appreciated that various other changes and modifications may be made to the embodiments described without departing from the spirit or scope of the invention.

1. A method of inhibiting myopia development in a human subject including the steps of:
prescribing a frequency and exposure time of a strobing or flickering light or pattern to reduce the rate of myopia development for the subject; and
treating the subject with a strobing or flickering light or pattern at the prescribed frequency and exposure time.

2. The method of claim 1 wherein the step of treating occurs each day or each alternate day.

3. The method of claim 1 further including the step of measuring the myopia of the subject.

4. A method of inhibiting myopia development in human subjects including the step of:
exposing the eyes of a person to light flashing at a frequency in the range of 1 to 60 Hz for a selected period.

5. The method of claim 4 wherein the step of exposing occurs each day or each alternate day.

6. The method of claim 4 further including the step of selecting the wavelength of the light, the intensity of the light, the frequency of flashing and the duration of flashing.

7. The method of claim 4 further including the step of recording feedback and using a feedback loop to adjust the treatment response to the effectiveness of the treatment in terms of measured progress of the subject.

8. The method of claim 4 wherein the light flashes at a frequency in the range between 5 and 20 Hz.

9. The method of claim 4 wherein the step of exposing is applied for at least 5 minute periods every hour over a 2 to 10 hour period.

10. The method of claim 4 wherein the step of exposing is applied for 10 minute periods every hour over a 2 to 10 hour period.

11. The method of claim 4 wherein the step of exposing is applied for at least 20 minute periods every hour over a 2 to 10 hour period.

12. The method of claim 4 wherein the step of exposing is applied during daylight hours.

13. The method of claim 4 wherein the light is visible light.

14. An apparatus for inhibiting myopia development in humans comprising:
a strobable light;
a means of adjusting a frequency at which the light strobes;
a means of adjusting a period of time over which the light strobes;
wherein said light strobes at a desired frequency for a desired time period.

15. The apparatus of claim 14 further comprising:
a feedback means of measuring myopia and making an adjustment to the period of time and the frequency the light strobes in response to the measured myopia.

16. The apparatus of claim 14 wherein the light is in the visible range.

17. The apparatus of claim 14 further comprising means for adjusting a wavelength of said strobable light.

18. The apparatus of claim 17 wherein the wavelength of the light is about 550 nm.

19. The apparatus of claim 14 wherein the strobable light operates at a frequency in the range 1 to 60 Hz.

20. The apparatus of claim 14 wherein the strobable light operates at a frequency in the range 5 to 20 Hz.

21. The apparatus of claim 14 wherein the frequency of the strobable light compensates for the frequency of the background lighting.

22. The apparatus of claim 14 wherein the intensity of the strobable light compensates for the intensity of the background lighting.

23. The apparatus of claim 14 wherein the wavelength of the strobable light compensates for the wavelength of the background light.

24. The apparatus of claim 14 further comprising a base.

25. The apparatus of claim 24 wherein the base is in the form of eyeglass frames with the light located near the hinge.

26. The apparatus of claim 24 wherein the base is mountable to actable.

27. The apparatus of claim 24 wherein the base is in the form of a lamp stand.

28. An apparatus for inhibiting myopia development in humans comprising:
a flickering pattern of low luminance and high luminance regions;
a means of adjusting a frequency at which the pattern flickers; and
a means of adjusting a period of time over which the pattern flickers;
wherein said pattern flickers at a desired frequency for a desired time period.

29. The apparatus of claim 28 comprising a television frequency signal generator that delivers a television frequency signal of the flickering pattern.

30. The apparatus of claim 28 comprising a computer when programmed to display the flickering pattern on a monitor or screen of said computer.
